SEQUENCE LISTING (21 sequences)

SEQ ID NO: 1 - RNA, 12 nt, Artificial Sequence
  Synthetic (Essential part of crRNA)
guuuuagagcua

SEQ ID NO: 2 - RNA, 10 nt, Artificial Sequence
  Synthetic (3′-terminal part of crRNA)
ugcuguuuug

SEQ ID NO: 3 - RNA, 60 nt, Artificial Sequence
  Synthetic (Essential part of tracrRNA)
uagcaaguuaaaauaaggcuaguccguuaucaacuugaaaaaguggcaccgagucggugc

SEQ ID NO: 4 - protein, 1368 aa, Artificial Sequence [sequence omitted]

SEQ ID NO: 5 - RNA, 14 nt, Artificial Sequence
  Synthetic (General formula of SpCas9 crRNA)
  misc_feature (1): n is targeting sequence comprising 18-22 or 20 nucleotides, each of which is A, U, C, or G
  misc_feature (14): n comprises 8-12 or 10 nucleotides, each of which is A, U, C, or G
nguuuuagagcuan

SEQ ID NO: 6 - RNA, 61 nt, Artificial Sequence
  Synthetic (General formula of SpCas9 tracrRNA)
  misc_feature (1): n comprises 6-20 or 8-19 nucleotides, each of which is A, U, C, or G
nuagcaaguuaaaauaaggcuaguccguuaucaacuugaaaaaguggcaccgagucggugc

SEQ ID NO: 7 - RNA, 80 nt, Artificial Sequence
  Synthetic (General formula of SpCas9 sgRNA)
  misc_feature (1): n is targeting sequence comprising 18-22 or 20 nucleotides, each of which is A, U, C, or G
  misc_feature (14): n is a linker comprising 3-5 or 4 nucleotides, each of which is A, U, C, or G
nguuucaguugcunaugcucuguaaucauuuaaaaguauuuugaacggaccucuguuugacacgucugaauaacuaaaaa

SEQ ID NO: 8 - RNA, 22 nt, Artificial Sequence
  Synthetic (General formula of Cpf1 crRNA)
  misc_feature (1): n is absent, U, A, or G
  misc_feature (2): n is A, or G
  misc_feature (5): n is U, A, or C
  misc_feature (12): n is absent, G, C, or A
  misc_feature (13): n is absent, A, U, C, or G
  misc_feature (14): n is U, G, or C
  misc_feature (15): n is U or G
  misc_feature (22): n is a targeting sequence including 15 to 30 nucleotides, each of which is A, U, C, or G
nnaunucuacunnnnguagaun

SEQ ID NO: 9 - DNA, 1758 bp, Artificial Sequence
  Synthetic (HygR-sfGFP)
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggagactagaatgtctaagggcgaggaactgttcaccggcgtggtgcccatcctggtggaactggatggcgacgtgaacggccacaagttctctgtgcggggagagggcgaaggcgacgccacaaatggcaagctgaccctgaagttcatctgcaccaccggcaagctgcccgtgccttggcctaccctcgtgaccacactgacctacggcgtgcagtgcttcagcagataccccgaccacatgaagcggcacgatttcttcaagagcgccatgcccgagggctatgtgcaggaacggaccatcagcttcaaggacgacggcacctacaagaccagagccgaagtgaagttcgagggcgacaccctcgtgaaccggatcgagctgaagggcatcgacttcaaagaggacggcaacatcctgggccacaagctggagtacaacttcaacagccacaacgtgtacatcaccgccgacaagcagaagaacggcatcaaggccaacttcaagatccggcacaacgtggaagatggcagcgtgcagctggccgaccactaccagcagaacacccccatcggagatggccccgtgctgctgcccgacaaccactacctgagcacccagagcgtgctgagcaaggaccccaacgagaagcgggaccacatggtgctgctggaatttgtgaccgccgctggcatcacccacggcatggacgagctgtacaagtctagttga

SEQ ID NO: 10 - DNA, 588 bp, Artificial Sequence
  Synthetic (CMV(Cytomegalovirus) promoter)
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagat

SEQ ID NO: 11 - DNA, 133 bp, Artificial Sequence
  Synthetic (SV40 intron)
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 12 - DNA, 75 bp, Artificial Sequence
  Synthetic (signal peptide coding sequence)
atggagcgcagtggctgggcccggcagacttttctcctagcgctgttgctgggggcgacgctgagagcgcgcgcg

SEQ ID NO: 13 - DNA, 24 bp, Artificial Sequence
  Synthetic (FLAG tag)
gattacaaggatgacgatgacaag

SEQ ID NO: 14 - DNA, 23 bp, Artificial Sequence
  Synthetic (RELN_sg1)
gcgctaggaggaaagtctgccgg

SEQ ID NO: 15 - DNA, 23 bp, Artificial Sequence
  Synthetic (RELN_sg2)
tttcctcctagcgctgttgctgg

SEQ ID NO: 16 - DNA, 23 bp, Artificial Sequence
  Synthetic (RELN_sg3)
ttcctcctagcgctgttgctggg

SEQ ID NO: 17 - DNA, 23 bp, Artificial Sequence
  Synthetic (RELN_sg4)
tcctcctagcgctgttgctgggg

SEQ ID NO: 18 - DNA, 23 bp, Artificial Sequence
  Synthetic (RELN_sg5)
cccccagcaacagcgctaggagg

SEQ ID NO: 19 - DNA, 23 bp, Artificial Sequence
  Synthetic (RELN_sg6)
tgttgctgggggcgacgctgagg

SEQ ID NO: 20 - DNA, 23 bp, Artificial Sequence
  Synthetic (RELN_sg7)
gttgctgggggcgacgctgaggg

SEQ ID NO: 21 - DNA, 9172 bp, Artificial Sequence
  Synthetic (full sequence of pAAY-hygro-sfGFP plasmid)
gagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggataccccgaagagtgagtttgccaagcagtcaccccacagttggaggagaatccacccaaaaggcagcctggtagacagggctggggtggcctctcgtggggtccaggccaagtaggtggcctggggcctctgggggatgcaggggaagggggatgcaggggaacggggatgcaggggaacggggctcagtctgaagagcagagccaggaacccctgtagggaaggggcaggagagccaggggcatgagatggtggacgaggaagggggacagggaagcctgagcgcctctcctgggcttgccaaggactcaaacccagaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacagccgcgtcagagcagctcaggttctgggagagggtagcgcagggtggccactgagaaccgggcaggtcacgaattcgagctcggtacccggggatccttcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggccagtggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcaactagacttgtacagctcgtccatgccgtgggtgatgccagcggcggtcacaaattccagcagcaccatgtggtcccgcttctcgttggggtccttgctcagcacgctctgggtgctcaggtagtggttgtcgggcagcagcacggggccatctccgatgggggtgttctgctggtagtggtcggccagctgcacgctgccatcttccacgttgtgccggatcttgaagttggccttgatgccgttcttctgcttgtcggcggtgatgtacacgttgtggctgttgaagttgtactccagcttgtggcccaggatgttgccgtcctctttgaagtcgatgcccttcagctcgatccggttcacgagggtgtcgccctcgaacttcacttcggctctggtcttgtaggtgccgtcgtccttgaagctgatggtccgttcctgcacatagccctcgggcatggcgctcttgaagaaatcgtgccgcttcatgtggtcggggtatctgctgaagcactgcacgccgtaggtcagtgtggtcacgagggtaggccaaggcacgggcagcttgccggtggtgcagatgaacttcagggtcagcttgccatttgtggcgtcgccttcgccctctccccgcacagagaacttgtggccgttcacgtcgccatccagttccaccaggatgggcaccacgccggtgaacagttcctcgcccttagacattctagtctcccccatctcccgatccggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcggcacgctgttgacgctgttaagcgggtcgctgcagggtcgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgtttaaacaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccagggtacccgatcgggatccaccggtcgtctcgccggggcagggggcgaattcgtcgacctcgaggccgccatggcagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggctgcacaccatcaccatcaccatcaccatcaccattaggcggccgcggctagcagcggccgcacagagacatctcaggtagcaccagcatagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggattaagggcgcgattaggatcttcctagagcatggctacgtagataagtagcatggcgggttctagagtcgacctgcaggcatgcaagcttgaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtccctagtggccccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2024/0229096 A1
(43) Pub. Date: Jul. 11, 2024

(54) RECOMBINANT CELL AND METHOD FOR PRODUCING ENDOGENOUS POLYPEPTIDE

(71) Applicant: HUB Biotech Co., Ltd., Seoul (KR)

(72) Inventors: Sang Su BAE, Seoul (KR); Jae Sung WOO, Seoul (KR); Ji Hyeon YU, Seoul (KR)

(21) Appl. No.: 16/760,167

(22) PCT Filed: Oct. 12, 2018
(86) PCT No.: PCT/KR2018/012051

§ 371 Date: Feb. 17, 2021

(30) Foreign Application Priority Data
Oct. 31, 2017 (KR) 10-2017-0143973

(51) Int. Cl. C12P 21/00; C12N 5/071; C12N 9/22; C12N 15/11; C12N 15/90

(52) U.S. Cl. CPC C12P 21/005; C12N 5/0686; C12N 9/22; C12N 15/11; C12N 15/907; C12N 2310/20; C12N 2800/80

(57) ABSTRACT

Provided are a composition for expressing and/or producing a polypeptide of interest in an animal cell, comprising a target-specific endonuclease system or an encoding gene thereof and a donor DNA structure; a recombinant animal cell into which said composition is introduced and a manufacturing method thereof; and a method for expressing and/or producing a polypeptide of interest in an animal cell, comprising a step of introducing said composition into the animal cell and/or a step of culturing said animal cell.

### TECHNICAL FIELD

Provided are a composition for expressing and/or producing a polypeptide of interest in animal cells, the composition comprising a target-specific endonuclease system or a gene coding therefor and a donor DNA structure; a recombinant animal cell having the composition introduced thereinto and a preparing method therefor, and a method for expressing and/or producing a polypeptide of interest in an animal cell, the method comprising a step of introducing the composition into the animal cells and/or a step of culturing the animal cells.

### BACKGROUND ART

As a rule, the mass production of a proteins found in an animal cell follow the modus operandi in which a plasmid DNA carrying a coding sequence (CDS) for the protein of interest is constructed through genetic recombination and then transfected into cells. This approach comprises the steps of synthesizing complementary DNA (cDNA) from mRNA encoding the protein of interest with reverse transcriptase, amplifying the same, and inserting the amplicon in a recombinant manner into a plasmid for expression in animal cells such as human cells. For effective extraction/purification of a protein of interest expressed in cells, the protein may be linked to a tag, such as an antibody, through recombination with the plasmid which is introduced into the cells.

However, the protein production by introducing a recombinant plasmid into cells suffers from the following problems:
1) difficulty in increasing cell culture scales and cell densities;
2) temporal limitation that the proteins should be extracted at particular times due to the temporary protein expression within cells;
3) plausibility of imparting a misstructure and/or malfunction to the protein upon introduction into heterogenous cells due to a difference in post-translational modifications such as folding, glycosylation, etc. therein.
4) difficulty in cloning into a plasmid a CDS coding for a protein having a large size or repeated sequences because the CDS becomes long in length.

In order to overcome such problems, therefore, there is a need for a technology for mass production of proteins that are structurally and functionally accurate in cells.

### PRIOR ART DOCUMENT

(Non-patent literature 0001) Andrianantoandro E et al. Mol Syst Biol. 2: 2006.0028 (2006)

### DISCLOSURE

### Technical Problem

An embodiment provides a composition for expressing a polypeptide of interest in an animal cell, the composition comprising a target-specific endonuclease system or a donor DNA structure.

Another embodiment provides a recombinant cell prepared by introducing the composition for expressing and/or producing a polypeptide of interest into an animal cell. The recombinant cell may be a cell in which the donor DNA structure contained in the composition is inserted to the 5′ end side of a gene coding for an endogenous polypeptide of interest in the genome. The recombinant cell may be used for producing the polypeptide of interest.

Another embodiment provides a recombinant cell wherein the donor DNA structure is inserted to a 5′ end side of a gene coding for an endogenous polypeptide of interest within a genome of the animal cell (host cell). The recombinant cell may be used for producing the polypeptide of interest. The donor DNA structure may comprise an exogenous promoter derived from a cell heterogenous to the host cell.

Another embodiment provides a composition for expressing a polypeptide of interest in an animal cell, or a composition for producing a polypeptide of interest in an animal cell, the composition comprising the recombinant cell.

Another embodiment provides a method for preparation of an animal cell for producing a polypeptide of interest, the method comprising a step of introducing the composition for producing the polypeptide of interest into the animal cell.

Another embodiment provides a method for expressing a polypeptide of interest in an animal cell, the method comprising a step of introducing the composition for expressing the polypeptide of interest into the animal cell. In the expression method, the animal cell that has the expression composition introduced thereinto is higher than animal cells that do not in terms of the expression level of the polypeptide of interest.

Another embodiment provides a method for producing a polypeptide of interest in an animal cell, the method comprising a step of introducing the polypeptide of interest into the animal cell. In the production method, the animal cell that has the expression composition introduced thereinto is higher than animal cells that do not in terms of the production level of the polypeptide of interest.

Another embodiment provides a method for producing a polypeptide of interest in an animal cell, the method comprising a step of culturing the recombinant cell for producing the polypeptide of interest.

The methods for expressing and/producing a polypeptide of interest in an animal cell are characterized by the absence of any step of externally introducing a coding sequence (CDS) for a polypeptide of interest into the animal cell.

### Technical Solution

Provided in this disclosure are a composition for producing a polypeptide of interest in an animal cell, the composition comprising a target-specific endonuclease system or a coding gene therefor and a donor DNA structure; a recombinant animal cell having the composition introduced thereinto; and a method for producing a polypeptide of interest in an animal cell, the method comprising introducing the composition into the animal cell. The polypeptide of interest may be an endogenous polypeptide encoded by the genome of the cell. The composition for producing a polypeptide of interest, the recombinant animal cell, and the method for producing polypeptide of interest, provided in the present disclosure, are characterized in that the endogenous polypeptide can be produced in a large amount in an animal cell while retaining the intact structure and/or function thereof, without recombinant introduction of a coding sequence (CDS) therefor.

Unless stated otherwise, the “donor DNA structure” does not contain a coding sequence (CDS) for a polypeptide of interest to be expressed.

An embodiment provides a composition for expressing a polypeptide of interest in an animal cells, the composition comprising a target-specific endonuclease system and a donor DNA structure.

Another embodiment provides a recombinant cell prepared by introducing the composition for expressing a polypeptide of interest into an animal cell. The recombinant cell is a cell in which the donor DNA structure contained in the composition is inserted to the 5′ end side of a gene coding for the endogenous polypeptide of interest within the genome of the cell. The recombinant cell may be used for producing the polypeptide of interest.

Another embodiment provides a recombinant cell in which the donor DNA structure is inserted to the 5′ end side of a gene coding for a polypeptide of interest in the genome of the animal cell (host cell) (e.g., between the initial codon and the endogenous promoter or 5′-UTR in the target gene). The recombinant cell may be used for producing the polypeptide of interest. The donor DNA structure may comprise a promoter derived from the host cells and an exogenous promoter derived from a cell heterogenous to the host cell.

Another embodiment provides a composition for producing a polypeptide of interest in an animal cell, the composition comprising either the composition for expressing the polypeptide of interest in the animal cell, or the recombinant cell.

Another embodiment provides a method for producing a recombinant cell for producing a polypeptide of interest, the method comprising a step of introducing the composition for producing the polypeptide of interest into an animal cell.

Another embodiment provides a method for expressing a polypeptide of interest in an animal cell, the method comprising a step of introducing the composition for expressing a polypeptide of interest into the animal cell. In the expression method, the animal cell that has the expression composition introduced thereinto is higher than animal cells that do not in terms of the expression level of the polypeptide of interest.

Another embodiment provides a method for producing a polypeptide of interest in an animal cell, the method comprising a step of introducing the polypeptide of interest into the animal cell. In the production method, the animal cell that has the expression composition introduced thereinto is higher than animal cells that do not in terms of the production level of the polypeptide of interest.

Another embodiment provides a method for producing a polypeptide of interest in an animal cell, the method comprising a step of culturing the recombinant cell for producing the polypeptide of interest.

Another embodiment provides a method for producing a polypeptide of interest in an animal cell, the method comprising the steps of: introducing into the animal cell a composition for producing the polypeptide of interest to prepare a recombinant cell in which the composition for producing the polypeptide of interest is inserted (introduced) to the 5′ end side of a gene coding for the polypeptide of interest within the genome (for example, between an initiation codon of the target gene and an endogenous promoter or 5′-UTR); and culturing the recombinant cell.

The methods for expressing and/producing a polypeptide of interest in an animal cell are characterized by the absence of any step of externally introducing a coding sequence (CDS) for a polypeptide of interest into the animal cell. Therefore, the methods do not need the fabrication of any additional CDS for the polypeptide of interest.

The method for producing a polypeptide of interest in an animal cell may further comprise a step of isolating (extracting) and/or purifying the polypeptide of interest, subsequent to the step of introducing a composition for expressing the polypeptide of interest and/or the step of culturing the recombinant animal cell for producing the polypeptide of interest.

Below, a detailed description will be given of the present disclosure.

As used herein, the term “polypeptide of interest” refers to a polypeptide to be produced and is intended to encompass any protein or peptide encoded by the genome of a host cell or expressed in the host cell, covering polypeptides unknown for activity thereof as well as proteins and/or peptides having desired activities in vivo (e.g., activities of preventing, alleviating, and/or treating specific diseases, or of substituting for biologically necessary substances). The polypeptide of interest may be at least one selected from the group consisting of polypeptides located in the cytoplasm, polypeptides located in the cell membrane, and extracellularly excreted polypeptides. For example, the polypeptide of interest may be at least one selected from the group consisting of enzymes, hormones, growth factors, receptors, transport polypeptides, immune polypeptides (generic name for polypeptides produced in immune cells), signaling polypeptides, and biologically structural polypeptides.

In an embodiment, the polypeptide of interest may be at least one selected from the group consisting of:
enzymes including hydrolysis enzymes (e.g., proteinases, phosphatases, etc.), oxidoreductases, enzymes of transferring a methyl group, a phosphoryl group, etc. (e.g., kinases),
hormones including insulin, growth hormones, growth hormone releasing hormones, melatonin, serotonin, thyroid hormone, thyrotropin-releasing hormone, epinephrine, norepinephrine, dopamine, adiponectin, adrenocorticotrophic hormone, adrenocorticotrophic hormone-releasing hormone, vasopressin, calcitonin, cholecystokinin, follicle-stimulating hormone, gastrin, ghrelin, glucagon, human chorionic gonadotropin, luteinizing hormone, paratormone, prolactin, secretin, lipotropin, and histamine,
growth factors including insulin-like growth factors (IGFs), epidermal growth factor (EGF), vascular growth factors (vascular endothelial growth factor (VEGF), angiopoietin, etc.), nerve growth factor (NGF), erythropoietin (EPO), fibroblast growth factor (FGF), platelet-derived growth factor (PDGF), transforming growth factor (TGF), growth/differentiation factor (GDF, e.g., GDF15), etc.,
receptors including G protein-coupled receptor (GPCR), receptor tyrosine kinase (RTK), ionotropic receptors, etc.,
transport polypeptides including hemoglobin, transferrin, etc.
immune polypeptides including immunoglobulins (e.g., IgG (IgG1, IgG2, IgG3, IgG4), IgA, IgD, IgM, IgE, etc.), cytokines (e.g., interleukins such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, etc.), interferon (IFN)-alpha, -beta, -gamma, -omega, or -tau, tumor necrosis factors such as TNF-alpha, -beta or -gamma, TNF-related apoptosis-inducing ligand (TRAIL), colony stimulating factor (CSF) such as granulocyte-colony stimulating factor (G-CSF), granulocyte-macrophage colony-stimulating factor (GM-CSF), macrophage colony-stimulating factor (M-CSF), etc.,
various signaling polypeptides such as extracellular matrix glycoproteins (e.g., Reelin, etc.), bone morphogenetic protein (BMP), etc., and
other biologically structural polypeptides such as collagen, elastin, keratin, tubulin, actin, fibrin, myosin, albumin, histone, casein, ovalbumin, etc.

As for a gene coding for a polypeptide of interest (hereinafter referred to “target gene”), its concrete contents (e.g., nucleic acid sequences, etc.) could be obvious to a person skilled in the art in view of the aforementioned definition thereof.

The polypeptide of interest may be a structure in which two or more amino acid residues are sequentially linked via a peptidyl bond and may include 2 to 10,000 amino acids, 2 to 9,000 amino acids, 2 to 8,000 amino acids, 2 to 7,000 amino acids, 2 to 6,000 amino acids, 2 to 5,000 amino acids, 2 to 4,000 amino acids, 2 to 3,000 amino acids, 2 to 2,000 amino acids, 2 to 1,000 amino acids, 50 to 10,000 amino acids, 50 to 9,000 amino acids, 50 to 8,000 amino acids, 50 to 7,000 amino acids, 50 to 6,000 amino acids, 50 to 5,000 amino acids, 50 to 4,000 amino acids, 50 to 3,000 amino acids, 50 to 2,000 amino acids, 50 to 1,000 amino acids, 100 to 10,000 amino acids, 100 to 9,000 amino acids, 100 to 8,000 amino acids, 100 to 7,000 amino acids, 100 to 6,000 amino acids, 100 to 5,000 amino acids, 100 to 4,000 amino acids, 100 to 3,000 amino acids, 100 to 2,000 amino acids, 100 to 1,000 amino acids, 500 to 10,000 amino acids, 500 to 9,000 amino acids, 500 to 8,000 amino acids, 500 to 7,000 amino acids, 500 to 6,000 amino acids, 500 to 5,000 amino acids, 500 to 4,000 amino acids, 500 to 3,000 amino acids, 500 to 2,000 amino acids, 500 to 1,000 amino acids, 1000 to 10,000 amino acids, 1000 to 9,000 amino acids, 1000 to 8,000 amino acids, 1000 to 7,000 amino acids, 1000 to 6,000 amino acids, 1000 to 5,000 amino acids, 1000 to 4,000 amino acids, 1000 to 3,000 amino acids, 1000 to 2,000 amino acids, 2000 to 10,000 amino acids, 2000 to 9,000 amino acids, 2000 to 8,000 amino acids, 2000 to 7,000 amino acids, 2000 to 6,000 amino acids, 2000 to 5,000 amino acids, 2000 to 4,000 amino acids, 2000 to 3,000 amino acids, 3000 to 10,000 amino acids, 3000 to 9,000 amino acids, 3000 to 8,000 amino acids, 3000 to 7,000 amino acids, 3000 to 6,000 amino acids, 3000 to 5,000 amino acids, or 3000 to 4,000 amino acids.

The polypeptide of interest may be an endogenous polypeptide. The endogenous polypeptide may refer to a polypeptide that is encoded by a gene in the genome of a host cell and expressed in the host cell, without introduction of an exogenous gene into the host cell.

The approach using an endogenous polypeptide enjoys the following advantages: (1) Because post-translational modifications such as folding, glycosylation, etc. are carried out in the cell itself, the genetic context of the cell can be utilized as it is, which is advantageous for the endogenous polypeptide to retain the native secondary and/or tertiary structures and/or functions thereof, compared to that expressed in the host cell into which a foreign gene has been inserted; and (2) even a polypeptide that is difficult to insert into a plasmid due to the size limitation of the coding sequence (CDS) therefor can be available because the insertion of an exogenous gene is not required.

The endogenous polypeptide may be a polypeptide derived from a eukaryotic animal, for example, primates such as humans, monkeys, marmosets, etc., carnivora animals such as dogs, cats, etc., artiodactyla animals such as pigs, cattle, sheep, etc., or birds such as chickens, ducks, etc. In addition, the host cell may be a eukaryotic animal cell from which the polypeptide to be produced is derived, for example, a cell from mammals including primates such as humans, monkeys, marmosets, etc., carnivora animals such as dogs, cats, etc., artiodactyla animals such as pigs, cattle, sheep, etc. or from birds such as chickens, ducks, etc. The cell may be an isolated cell from a living entity.

As used herein, the term “target-specific endonuclease system”, also called programmable nuclease, refers to a functional unit that can recognize and cleave a specific target nucleic acid sequence and may comprise: an endonuclease, a nucleic acid molecule coding therefor (first nucleic acid molecule), or a recombinant vector carrying the nucleic acid molecule (first recombinant vector); and a nucleic acid molecule recognizing a specific target nucleic acid sequence (DNA or RNA; second nucleic acid molecule) or a recombinant vector carrying the nucleic acid molecule (second recombinant vector).

The endonuclease may be any target-specific endonuclease that has the activity of cleaving a specific gene site on single and/or double strands and can act, together with a nucleic acid molecule recognizing a specific nucleic acid sequence, to cleave a specific target gene sequence.

For example, the target-specific nuclease may be at least one selected from the group consisting of:
transcription activator-like effector nuclease (TALEN) in which a transcription activator-like (TAL) effector DNA-binding domain, derived from a gene responsible for plant infection, for recognizing a specific target sequence, is fused to a DNA cleavage domain;
zinc-finger nuclease (ZFN);
RNA-guided engineered nuclease (RGEN), which is derived from the microbial immune system CRISPR, such as Cas proteins (e.g., Cas9, etc.), Cpf1, and the like; and
DNA-guided endonuclease (e.g., Ago homolog), but is not limited thereto.

The target-specific endonuclease recognizes specific base sequences in the genome of prokaryotic cells and/or animal and plant cells (i.e., eukaryotic cells), including human cells, to cause double strand breaks (DSBs) or single strand breaks (SSBs). The double strand breaks create a blunt end or a cohesive end by cleaving the double strands of DNA. DSBs are efficiently repaired by homologous recombination or non-homologous end-joining (NHEJ) mechanisms within the cell, which allows the introduction of desired mutations (substitutions, deletions, and insertions in part or entirety of a gene) into on-target sites during this process.

In an embodiment, the target-specific endonuclease may be RNA-guided engineered endonuclease (RGEN) derived from CRISPR. In this regard, the target-specific endonuclease system may comprise:
(1) RNA-guided endonuclease, a nucleic acid molecule coding therefor, or a recombinant vector carrying the nucleic acid molecule; and
(2) a guide RNA capable of hybridizing with (or having a nucleic acid sequence complementary to) a target nucleic acid sequence, a DNA coding therefor, or a recombinant vector carrying the DNA.

In one embodiment, the target-specific endonuclease may be at least one selected from the group consisting of endonucleases included in the type II and/or type V CRISPR system, such as Cas proteins (e.g., Cas9 protein (CRISPR (clustered regularly interspaced short palindromic repeats) associated protein 9)), Cpf1 protein (CRISPR from Prevotella and Francisella 1), etc. The guide RNA plays a role in guiding the target-specific endonuclease to a specific target site on a genomic DNA. The RNA-guided endonuclease and the guide RNA are associated with each other ex vivo (in vitro) to form a ribonucleic acid-protein complex (RNA-Guided Engineered Nuclease) and as such, are introduced in a ribonucleoprotein (RNP) form into cells. Alternatively, nucleic acid molecules or DNAs coding for the RNA-guided endonuclease and the guide RNA are introduced into cells via respective plasmids or one plasmid, after which the RNA-guided endonuclease and the guide RNA are expressed and form a ribonucleoprotein, which is active within cells.

The Cas protein, which is a main protein component in the CRISPR/Cas system, accounts for activated endonuclease or nickase activity. The Cas protein or gene information may be obtained from a well-known database such as GenBank at the NCBI (National Center for Biotechnology Information).

By way of example, the Cas protein may be at least one selected from the group consisting of:
a Cas protein derived from Streptococcus sp., e.g., Streptococcus pyogenes (i.e., SwissProt Accession number Q99ZW2 (NP_269215.1); SEQ ID NO: 4);
a Cas protein derived from Campylobacter sp., e.g., Campylobacter jejuni;
a Cas protein derived from Streptococcus sp., e.g., Streptococcus thermophiles or Streptococcus aureus;
a Cas protein derived from Neisseria meningitidis;
a Cas protein derived from Pasteurella sp., e.g., Pasteurella multocida; and
a Cas protein derived from Francisella sp., e.g., Francisella novicida, but is not limited thereto.

In an embodiment where the Cas9 protein is derived from Streptococcus pyogenes, the PAM sequence may be 5′-NGG-3′ (N is A, T, G, or C) and cleavage may occur between the third and the forth nucleotide residue on the 5′ end of the PAM sequence while the guide RNA may hybridize with a consecutive 17 bp- to 23 bp-long, for example, 20 bp-long target nucleic acid region sequence located adjacent to the 5′- and/or 3′-end of the PAM sequence or a sequence on the complementary strand to the PAM sequence (in greater detail, the target nucleic acid sequence on a complementary strand to the strand on which the PAM sequence is located).

In another embodiment where the Cas9 protein is derived from Campylobacter jejuni, the PAM sequence may be 5′-NNNNRYAC-3′(N's are each independently A, T, C or G, R is A or G, and Y is C or T) and the guide RNA may hybridize with a consecutive 17 bp- to 23 bp-long, for example, 20 bp-long target nucleic acid sequence located adjacent to the 5′- and/or 3′-end of the PAM sequence or a sequence on a complementary strand to the PAM sequence.

In another embodiment where the Cas9 protein is derived from Streptococcus thermophiles, the PAM sequence may be 5′-NNAGAAW-3′ (N's are each independently A, T, C, or G, and W is A or T) and the guide RNA may hybridize with a consecutive 17 bp- to 23 bp-long, for example, 20 bp-long target nucleic acid sequence region located adjacent to the 5′- and/or 3′-end of the PAM sequence or a sequence on a complementary strand to the PAM sequence.

According to another embodiment, in a case where the Cas9 protein is derived from Neisseria meningitidis, the PAM sequence may be 5′-NNNNGATT-3′(N's are each independently A, T, C or G) and the guide RNA may hybridize with a consecutive 17 bp- to 23 bp-long, for example, 20 bp-long target nucleic acid sequence region located adjacent to the 5′- and/or 3′-end of the PAM sequence or a sequence on a complementary strand to the PAM sequence.

In another embodiment where the Cas9 protein is derived from Streptocuccus aureus, the PAM sequence may be 5′-NNGRR(T)-3′ (N's are each independently A, T, C or G, R is A or G, and (T) means an optional sequence included therein) and the guide RNA may hybridize with a consecutive 17 bp- to 23 bp-long, for example, 20 bp-long target nucleic acid sequence region located adjacent to the 5′- and/or 3′-end of the PAM sequence or a sequence on a complementary strand to the PAM sequence.

The Cpf1 protein, which is an endonuclease in a new CRISPR system distinguished from the CRISPR/Cas system, is small in size relative to Cas9, requires no tracrRNA, and can act with the guidance of single guide RNA. In addition, the Cpf1 protein recognizes a thymine-rich PAM (protospacer-adjacent motif) sequence and cleaves DNA double strands to form a cohesive end (cohesive double-strand break).

By way of example, the Cpf1 protein may be derived from Candidatus spp., Lachnospira spp., Butyrivibrio spp., Peregrinibacteria, Acidominococcus spp., Porphyromonas spp., Prevotella spp., Francisella spp., Candidatus Methanoplasma, or Eubacterium spp., e.g., from Parcubacteria bacterium (GWC2011_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum, Candidatus Paceibacter, Eubacterium eligens, etc., but is not limited thereto.

In a case where Cpf1 protein is used as the endonuclease, the PAM sequence is 5′-TTN-3′ (N is A, T, C, or G) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the 5′-TTN-3′ sequence in a target gene and the guide RNA may hybridize with a consecutive 17 bp- to 23 bp-long, for example, 21 bp- to 23 bp-long target nucleic acid sequence region located adjacent to the 5′- and/or 3′-end of the PAM sequence.

The target-specific endonuclease may be isolated from microbes or may be an artificial or non-naturally occurring enzyme as obtained by recombination or synthesis. For use, the target-specific endonuclease may be in the form of an mRNA pre-described or a protein pre-produced in vitro or may be included in a recombinant vector so as to be expressed in target cells or in vivo. In an embodiment, the target-specific endonuclease (e.g., Cas9, Cpf1, etc.) may be a recombinant protein made with a recombinant DNA (rDNA). The term “recombinant DNA” means a DNA molecule formed by artificial methods of genetic recombination, such as molecular cloning, to bring together homologous or heterologous genetic materials from multiple sources. For use in producing a target-specific endonuclease by expression in a suitable organism (in vivo or in vitro), for example, the recombinant DNA may have a nucleotide sequence that is reconstituted with optimal codons for expression in the organism, which are selected from codons coding for a protein to be produced.

The target-specific endonuclease used herein may be a mutant target-specific nuclease in an altered form. The mutant target-specific endonuclease may refer to a target-specific endonuclease mutated to lack the endonuclease activity of cleaving double strand DNA and may be, for example, at least one selected from among mutant target-specific endonucleases mutated to lack endonuclease activity but to retain nickase activity and mutant target-specific endonucleases mutated to lack both endonuclease and nickase activities. As such, the mutation of the target-specific endonuclease (e.g., amino acid substitution, etc.) may occur at least in the catalytically active domain of the nuclease (for example, RuvC catalyst domain for Cas9). In an embodiment, when the target-specific nuclease is a Streptococcus pyogenes-derived Cas9 protein (SwissProt Accession number Q99ZW2(NP_269215.1); SEQ ID NO: 4), the mutation may be amino acid substitution at one or more positions selected from the group consisting of a catalytic aspartate residue (e.g., aspartic acid at position 10 (D10) for SEQ ID NO: 4, etc.), glutamic acid at position 762 (E762), histidine at position 840 (H840), asparagine at position 854 (N854), asparagine at position 863 (N863), and aspartic acid at position 986 (D986) on the sequence of SEQ ID NO: 4. A different amino acid to be substituted for the amino acid residues may be alanine, but is not limited thereto.

In another embodiment, the mutant target-specific endonuclease may be a mutant that recognizes a PAM sequence different from that recognized by wild-type Cas9 protein. For example, the mutant target-specific endonuclease may be a mutant in which at least one, for example, all of the three amino acid residues of aspartic acid at position 1135 (D1135), arginine at position 1335 (R1335), and threonine at position 1337 (T1337) of the Streptococcus pyogenes-derived Cas9 protein are substituted with different amino acids to recognize NGA (N is any residue selected from among A, T, G, and C) different from the PAM sequence (NGG) of wild-type Cas9.

In one embodiment, the mutant target-specific endonuclease may have the amino acid sequence (SEQ ID NO: 4) of Streptococcus pyogenes-derived Cas9 protein on which amino acid substitution has been made for:
(1) D10 or H840;
(2) D1135, R1335, T1337, or D1135+R1335+T1337; or
(3) both of (1) and (2) residues.

As used herein, the term “a different amino acid” means an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all variants thereof, exclusive of the amino acid retained at the original mutation positions in wild-type proteins. In one embodiment, “a different amino acid” may be alanine, valine, glutamine, or arginine.

In another embodiment, the mutant target-specific endonuclease may have the following mutations (substitutions) for the amino acid sequence (Q99ZW2(NP_269215.1)) of Streptococcus pyogenes-derived Cas9 protein.

TABLE 1

| | |
|---|---|
| Cas9 | Mutations |

Cas9 mutant with reduced off-target effect

| | |
|---|---|
| Sniper Cas9 | F539S/M763I/K890N |
| evoCas9 | M495V/Y515N/K526E/R661Q |
| HypaCas9 | N692A/M694A/Q695A/H698A |
| HeFSpCas9 | N497A/R661A/Q695A/K848A/Q926A/K1003A/R1060A |
| SpCas9-HF | N497A/R661A/Q695A/Q926A |
| eSpCas9 (1.1) | K848A/K1003A/R1060A |

Cas9 mutant recognizing other PAM sequence

| | |
|---|---|
| xCas9-3.7 | A262T/R324L/S409I/E480K/E543D/M694I/E1219V |

As used herein, the term “guide RNA” refers to an RNA that includes a targeting sequence capable of hybridizing with a specific nucleic acid sequence (target nucleic acid sequence) of the genome in a cell and functions to associate with an RNA-guided endonuclease, such as Cas proteins, Cpf1, etc. in vitro or in vivo (or in cells) and to guide the endonuclease to a target gene (or target site). When being introduced into a host cell, the guide RNA may be in the form of RNA or DNA coding therefor and may be or may not be associated with an RNA-guided endonuclease.

The guide RNA may be suitably selected depending on kinds of the endonuclease to be complexed therewith and/or origin microorganisms thereof.

For example, the guide RNA may be at least one selected from the group consisting of:
CRISPR RNA (crRNA) including a region (targeting sequence) capable of hybridizing with a target nucleic acid sequence;
trans-activating crRNA (tracrRNA) including a region interacting with a nuclease such as Cas protein, Cpf1, etc.; and
single guide RNA (sgRNA) in which main regions of crRNA and tracrRNA (e.g., a crRNA region including a targeting sequence and a tracrRNA region interacting with nuclease) are fused to each other.

In detail, the guide RNA may be a dual RNA including CRISPR RNA (crRNA) and trans-activating crRNA (tracrRNA) or a single guide RNA (sgRNA) including main regions of crRNA and tracrRNA.

The sgRNA may include a region (named “spacer region”, “target DNA recognition sequence”, “base pairing region”, etc.) having a complementary sequence (targeting sequence) to a target nucleic acid sequence, and a hairpin structure for binding to a Cas protein. In greater detail, the sgRNA may include a region having a targeting sequence, a hairpin structure for binding to a Cas protein, and a terminator sequence. These moieties may exist sequentially in the 5′ to 3′ direction, but without limitations thereto. So long as it includes main regions of crRNA and tracrRNA and a complementary sequence to a target DNA, any guide RNA can be used in the present disclosure.

For editing a target gene, for example, the Cas9 protein requires two guide RNAs, that is, a CRISPR RNA (crRNA) having a nucleotide sequence capable of hybridizing with a target site in the target gene and a trans-activating crRNA (tracrRNA) interacting with the Cas9 protein. In this context, the crRNA and the tracrRNA may be coupled to each other to form a crRNA:tracrRNA duplex or connected to each other via a linker so that the RNAs can be used in the form of a single guide RNA (sgRNA). In one embodiment, when a Streptococcus pyogenes-derived Cas9 protein is used, the sgRNA may form a hairpin structure (stem-loop structure) in which the entirety or a part of the crRNA having a hybridizable nucleotide sequence is connected to the entirety or a part of the tracrRNA including an interacting region with the Cas9 protein via a linker (responsible for the loop structure).

The guide RNA, specially, crRNA or sgRNA, includes a targeting sequence complementary to a target nucleic acid sequence and may contain one or more, for example, 1-10, 1-5, or 1-3 additional nucleotides at an upstream region of crRNA or sgRNA, particularly at the 5′ end of sgRNA or the 5′ end of crRNA of dual RNA. The additional nucleotide(s) may be guanine(s) (G), but are not limited thereto.

In another embodiment, when the nuclease is Cpf1, the guide RNA may include crRNA and may be appropriately selected, depending on kinds of the Cpf1 protein to be complexed therewith and/or origin microorganisms thereof.

Concrete sequences of the guide RNA may be appropriately selected depending on kinds of the nuclease (Cas9 or Cpf1) (i.e., origin microorganisms thereof) and are an optional matter which could easily be understood by a person skilled in the art.

In an embodiment, when a Streptococcus pyogenes-derived Cas9 protein is used as a target-specific nuclease, crRNA may be represented by the following General Formula 1:

(General Formula 1:: SEQ ID NO: 5)
5′-(N_{cas9})ₗ-(GUUUUAGAGCUA)-(X_{cas9})ₘ-3′

wherein:
N_{cas9} is a targeting sequence, that is, a region determined according to a target nucleic acid sequence (i.e., a sequence capable of hybridizing with a target nucleic acid sequence), 1 represents a number of nucleotides included in the targeting sequence and may be an integer of 15 to 30, 17 to 23 or 18 to 22, for example, 20;
the region including 12 consecutive nucleotides (GUUUUAGAGCUA; SEQ ID NO: 1) adjacent to the 3′-end of the targeting sequence is essential for crRNA;
X_{cas9} is a region including m nucleotides present on the 3′-end side of crRNA (that is, present adjacent to the 3′-end of the essential region); and
m may be an integer of 8 to 12, for example, 11 wherein the m nucleotides may be the same or different and are independently selected from the group consisting of A, U, C, and G.

In an embodiment, the X_{cas9} may include, but is not limited to, UGCUGUUUUG (SEQ ID NO: 2).

In addition, the tracrRNA may be represented by the following General Formula 2:

(General Formula 2: SEQ ID NO: 6)
5′-(Y_{cas9})ₚ-
(UAGCAAGUUAAAAUAAGGCUAGUCCGUUAUCAACUUGAAAAAGUGGCA
CCGAGUCGGUGC)-3′

wherein,
the region represented by 60 nucleotides (UAGCAAGUUAAAAUAAGGCUAGUCCGUUAUCAACUUGAAAAAGUGGCACCG AGUCGGUGC) (SEQ ID NO: 3) is essential for tracrRNA,
Y_{cas9} is a region including p nucleotides present adjacent to the 3′-end of the essential region, and
p is an integer of 6 to 20, for example, 8 to 19 wherein the p nucleotides may be the same or different and are independently selected from the group consisting of A, U, C, and G.

Furthermore, sgRNA may form a hairpin structure (stem-loop structure) in which a crRNA moiety including the targeting sequence and the essential region of the crRNA and a tracrRNA moiety including the essential region (60 nucleotides) of the tracrRNA are connected to each other via an oligonucleotide linker (responsible for the loop structure). In greater detail, the sgRNA may have a hairpin structure in which a crRNA moiety including the targeting sequence and an essential region of crRNA is coupled with the tracrRNA moiety including the essential region of tracrRNA to form a double-strand RNA molecule with connection between the 3′ end of the crRNA moiety and the 5′ end of the tracrRNA moiety via an oligonucleotide linker.

In one embodiment, the sgRNA may be represented by the following General Formula 3:

(General Formula 3: SEQ ID NO: 7)
5′-(N_{cas9})ₗ-(GUUUUAGAGCUA)-(oligonucleotide
linker)-(UAGCAAGUUAAAAUAAGGCUAGUCCGUUAUCAACUUGAAA
AAGUGGCACCGAGUCGGUGC)-3′

wherein (N_{cas9})ₗ is a targeting sequence defined as in General Formula 1.

The oligonucleotide linker included in the sgRNA may be 3-5 nucleotides long, for example 4 nucleotides long in which the nucleotides may be the same or different and are independently selected from the group consisting of A, U, C, and G.

The crRNA or sgRNA may further contain 1 to 3 guanines (G) at the 5′ end thereof (that is, the 5′ end of the targeting sequence of crRNA).

The tracrRNA or sgRNA may further comprise a terminator inclusive of 5 to 7 uracil (U) residues at the 3′ end of the essential region (60 nt long) of tracrRNA.

In another embodiment, when the endonuclease is a Cpf1 system, the guide RNA (crRNA) may be represented by the following General Formula 4:

(General Formula 4: SEQ ID NO: 8)
5′-n1-n2-A-U-n3-U-C-U-A-C-U-n4-n5-n6-n7-G-U-A-G-
A-U-(Ncpf1)q-3′

wherein, n1 is null or represents U, A, or G, n2 represents A or G, n3 represents U, A, or C, n4 is null or represents G, C, or A, n5 represents A, U, C, or G, or is null, n6 represents U, G, or C, n7 represents U or G, Ncpf1 is a targeting sequence including a nucleotide sequence capable of hybridizing with a target nucleic acid site and is determined depending on the target nucleic acid sequence, and q represents a number of nucleotides included therein and may be an integer of 15 to 30. The target sequence (hybridizing with crRNA) of the target gene is a 15 to 30 (e.g., consecutive) nucleotide-long sequence adjacent to the 3′ end of PAM (5′-TTN-3′ or 5′-TTTN-3′; N is any nucleotide selected from A, T, G, and C.

In General Formula 4, the 5 nucleotides from the 6th to the 10th position from the 5′ end (5′ end stem region) and the 5 nucleotides from the 15th (16th when n4 is not null) to the 19th (20th when n4 is not null) position from the 5′ end are complementary to each other in the antiparallel manner to form a duplex (stem structure), with the concomitant formation of a loop structure composed of 3 to 5 nucleotides between the 5′ end stem region and the 3′ end stem region.

For the Cpf1 protein, the crRNA (e.g., represented by General Formula 4) may further comprise 1 to 3 guanine residues (G) at the 5′ end.

In crRNA sequences for Cpf1 proteins available from microbes of Cpf1 origin, 5′ end sequences (exclusive of targeting sequence regions) are illustratively listed in Table 2:

TABLE 2

| | 5′ end sequence of guide RNA |
|---|---|
| Cpf1 source microorganism | (crRNA) (5′-3′) |
| Parcubacteria bacterium GWC2011_GWC2_44_17 | AAAUUUCUACU-UUUGUAGAU |
| (PbCpf1) | |
| Peregrinibacteria bacterium GW2011_GWA_33_10 | GGAUUUCUACU-UUUGUAGAU |
| (PeCpf1) | |
| Acidaminococcus sp. BVBLG (AsCpf1) | UAAUUUCUACU-CUUGUAGAU |
| Porphyromonas macacae (PmCpf1) | UAAUUUCUACU-AUUGUAGAU |
| Lachnospiraceae bacterium ND2006 (LbCpi1) | GAAUUUCUACU-AUUGUAGAU |
| Porphyromonas crevioricanis (PcCpf1) | UAAUUUCUACU-AUUGUAGAU |
| Prevotella disiens (PdCpf1) | UAAUUUCUACU-UCGGUAGAU |
| Moraxella bovoculi 237 (MbCpf1) | AAAUUUCUACUGUUUGUAGAU |
| Leptospira inadai (LiCpf1) | GAAUUUCUACU-UUUGUAGAU |
| Lachnospiraceae bacterium MA2020 (Lb2Cpf1) | GAAUUUCUACU-AUUGUAGAU |
| Francisella novicida U112 (FnCpf1) | UAAUUUCUACU-GUUGUAGAU |
| Candidatus Methanoplasma termitum (CMtCpf1) | GAAUCUCUACUCUUUGUAGAU |
| Eubacterium eligens (EeCpf1) | UAAUUUCUACU--UUGUAGAU |

(-: denotes the absence of any nucleotide)

The target nucleic acid sequence in a target gene to which the targeting sequence of the guide RNA is coupled (hybridized) can be represented by a sequence (1) of about 17 to about 23 or about 18 to about 22, for example, 20 consecutive nucleotides located adjacent to the 5′ and/or 3′ end of the protospacer adjacent motif (PAM) sequence of the target gene or a complementary sequence thereto (2). In this regard, a sequence to which the targeting sequence of the guide RNA is practically coupled may be the complementary sequence (2).

The target nucleic acid sequence in a target gene may be selected from nucleic acid sequences of the initiation codon region (e.g., 5′ end site of the initiation codon) in the target gene when the guide RNA (or targeting sequence) capable of hybridizing with any one nucleic acid sequence of the initiation codon region is remarkably low in hybridization with other nucleic acid sequences including three or less, two or less, or one mismatch, compared to the nucleic acid sequence (for example, upon gene editing with the guide RNA, DNA mutation is less than 1%, 0.5%, 0.1%, 0.05%, 0.01%, 0.005%, or 0.001%), or does not hybridize therewith.

As used herein, the term “nucleotide sequence” of the guide RNA refers to a nucleotide sequence having a sequence complementarity of 50% or higher, 60% or higher, 70% or higher, 80% or higher, 90% or higher, 95% or higher, 99% or higher, or 100% to a target nucleic acid sequence (that is, a nucleic acid sequence in a target nucleic acid region of the DNA strand on which the PAM sequence is located or in a target nucleic acid region of the complementary strand) and capable of complementarily binding to the nucleotide sequence of the complementary strand. Hereinafter, the term is used in the same meaning unless otherwise stated. The sequence homology can use a typical sequence comparison mean (e.g., BLAST, GCG (Genetics Computer Group, Madison Wis.) program package).

In the method, the transduction of the guide RNA and the RNA-guided endonuclease (e.g., Cas9 protein) into cells may be performed by directly introducing the guide RNA and the RNA-guided endonuclease into cells with the aid of a conventional technique (e.g., electroporation, etc.) or by introducing one vector (e.g., plasmid, viral vector, etc.) carrying both a guide RNA-encoding DNA molecule and a RNA-guided endonuclease-encoding gene or respective vectors carrying the DNA molecule and the gene into cells or through mRNA delivery.

The RNA-guided endonuclease (e.g., Cas9 protein) or a nucleic acid molecule coding therefor, the guide RNA or a DNA molecule coding therefor, or a vector carrying at least one of the nucleic acid molecules and the DNA molecules may be introduced (delivered) into cells, using a suitable one of well-known techniques such as microinjection, electroporation, DEAE-dextran treatment, lipofection, nanoparticle-mediated transduction, protein translocation domain (PTD)-mediated introduction, virus-mediated gene transfer, and PEG-mediated transfection, but without limitations thereto.

The RNA-guided endonuclease (e.g., Cas9 protein) may further include a pertinent nuclear localization signal (NLS) for the intranuclear translocation of the Cas9 protein within a host cell.

As used herein, the term “cleavage” means the breakage of the covalent backbone in a nucleic acid sequence. The cleavage includes enzymatic or chemical hydrolysis of a phosphodiester bond, but is not limited thereto, and may be performed by various other methods. Cleavage may be possible on both single strands and double strands. The cleavage of a double-strand may result from the cleavage of the two distinct single strands, with the consequent production of blunt ends or staggered ends.

As used herein, the term “donor DNA structure” refers to a donor DNA molecule to be inserted into the genome of a host cell or to a recombinant vector carrying the donor DNA molecule (third recombinant vector).

The donor DNA molecule comprises a promoter. The promoter, which is an exogenous promoter derived from a cell other than a host cell (a cell different from a host cell), may be at least one selected from all promoters capable of inducing the overexpression of a gene operatively linked thereto under the control of an expression system of the host cell. By way of example, the promoter may be at least one selected from a CMV promoter (e.g., a human CMV immediate-early promoter, a mouse CMV immediate-early promoter, etc.), a T7 promoter, an SP6 promoter, an rpr-1 promoter, an rrk promoter, a U6 promoter, a UBC promoter, an ACTB promoter, an EFIA promoter, a CAG promoter, an SV40 promoter, a PGK promoter, and a TRE promoter.

The promoter may be inserted to the 5′ end side of the initiation codon of a target gene coding for a polypeptide of interest in the genome of a host cell (e.g., between the initiation codon and an endogenous promoter or 5′-UTR in a target gene; the “initiation codon” may refer to the codon encoding the first amino acid of a polypeptide inclusive of a signal peptide or a polypeptide of interest exclusive of a signal peptide; hereinafter, the same meaning) in such a manner that the promoter is operatively linked to the target gene. As used herein, the term “operatively linked” refers to a functional linkage (cis) between the promoter and the target gene. When being “operatively linked” to a target gene, the promoter can regulate the transcription and/or translation of the target gene. For a functional linkage to a target gene, the promoter may be linked toward the 5′ end of the target gene.

The promoter may be inserted into a site cleaved by the target-specific endonuclease system stated above. Therefore, the nucleic acid molecule (e.g., guide RNA), included in the target-specific endonuclease system, for recognizing a specific target nucleic acid sequence may be designed to recognize a nucleic acid sequence in the vicinity of the initiation codon of a target gene as a target nucleic acid sequence (that is, the targeting sequence is designed to have a sequence hybridizable with (complementary to) the target nucleic acid sequence) so that the endonuclease can cleave the 5′ end side of the target gene coding for a polypeptide of interest within the genome of the host cell (e.g., a site between the initiation codon and the endogenous promoter or 5′-UTR in the target gene).

The promoter may be inserted at a predetermined position on the 5′ end side of the initiation codon of a target gene in the genome of the host cell and as such, can regulate the expression of the target gene operatively linked thereto instead of (in substitution for) the endogenous promoter. Irrespective of whether or not the promoter (exogenous promoter) inserted at a predetermined position to the 5′ end side of the initiation of the target gene in the genome of the host cell replaces an endogenous promoter in the genome of the host cell, the endogenous promoter loses the intrinsic function thereof and the target gene is expressed depending on the inserted promoter.

In another embodiment, the donor DNA molecule may further comprise at least one selected from the group consisting of a suitable tag encoding nucleic acid molecule (tag gene), a selection marker, a reporter gene, a nucleic acid molecule encoding a signal peptide for an endogenous polypeptide of interest, an exogenous intron (e.g., at least one selected from the group consisting of SV40 intron, CMV intron A, beta-globin intron, ubiquitin intron (UbC intron), and hGH intron) in addition to the exogenous promoter described above.

In an embodiment, the donor DNA molecule may essentially comprise the exogenous promoter stated above. In another embodiment, the donor DNA molecule may comprise a tag encoding nucleic acid sequence in addition to the exogenous promoter. In this regard, the exogenous promoter and the tag encoding nucleic acid sequence may be included in one DNA structure or in respective DNA structures. When the exogenous promoter and the tag encoding nucleic acid sequence are included in one DNA structure, the exogenous DNA molecule may comprise the exogenous promoter and the tag encoding nucleic acid sequence (gene) sequentially in the 5′ to 3′ direction. Furthermore, when the exogenous promoter and the tag encoding nucleic acid sequence are included in one DNA structure, the donor DNA molecule may further comprise a nucleic acid molecule encoding a signal peptide for an endogenous polypeptide of interest at the 5′ end of the tag encoding nucleic acid sequence in addition to the exogenous promoter and the tag encoding nucleic acid sequence (in this case, the donor DNA molecule may comprise an exogenous promoter, a nucleic acid molecule encoding a signal peptide for an endogenous polypeptide of interest, and a tag encoding nucleic acid sequence sequentially in the 5′ to 3 direction). In another embodiment, the donor DNA molecule may further comprise at least one selected from the group consisting of a selection marker and an exogenous intron (e.g., SV40 intron) in addition to the exogenous promoter, the tag encoding nucleic acid sequence, and the nucleic acid molecule encoding a signal peptide for an endogenous polypeptide of interest, linked to the 5′ end of the tag encoding nucleic acid sequence. In an embodiment, the donor DNA molecule may sequentially comprise (1) a selection marker, (2) an exogenous promoter, (3) an SV40 intron, (4) a nucleic acid molecule encoding a signal peptide for an endogenous polypeptide of interest, and (5) a tag encoding nucleic acid sequence in the 5′ to 3′ direction.

The tag is adapted to facilitate the isolation and/or purification of the endogenous polypeptide of interest produced in the host cell and may be linked to the N-end coding region (5′ end) or the C-end coding region (3′ end) of the DNA sequence or downstream of the nucleic acid molecule encoding a signal peptide for an endogenous polypeptide of interest (3′ end). In the case where the tag is linked downstream of the nucleic acid molecule encoding a signal peptide for an endogenous polypeptide of interest, the signal peptide is removed in the expression process of the polypeptide of interest in a host cell to expose the N-end of the tag, which makes it possible to easily detect and/or purify the endogenous polypeptide of interest with a substance capable of specifically binding to the tag (e.g., an antibody). The tag may be bound to an antibody and may be any typical tag so long as it can generate a signal such as fluorescence, luminescence, a color, etc. For example, the tag may be at least one selected from the group consisting of c-myc, 6×His, FLAG, HA, V5, and TAP, but is not limited thereto.

The nucleic acid molecule encoding a signal peptide for an endogenous polypeptide of interest may be a nucleic acid sequence artificially obtained (synthesized) ex vivo. When the donor DNA structure comprises a nucleic acid molecule encoding a signal peptide for an endogenous polypeptide of interest, the target-specific endonuclease system may be designed to cleave the 3′ end or an inner site of the nucleic acid sequence encoding the signal peptide in the amino acid sequence of the endogenous polypeptide of interest. For example, the target-specific endonuclease system may be designed to cleave a site adjacent to the 5′ end of the codon encoding the first amino acid in the amino sequence of the endogenous polypeptide of interest except for the signal peptide.

The selection marker is a gene functioning to guide the selection of a host cell into which the donor DNA structure has been inserted. For example, the selection marker may be at least one selected from the group consisting of a drug resistance marker, a fluorescent marker, a luminescent marker, a metabolism-related marker, and a gene amplification marker, but is not limited thereto. The fluorescent marker may be at least one selected from genes coding for fluorescent proteins (e.g., green fluorescent protein (GFP), cyan fluorescent protein (CFP), yellow fluorescent protein (YFP), red fluorescent protein (dsRFP), etc.), but is not limited thereto. The luminescent marker may be at least one selected from the group consisting of genes coding for luminescent proteins such as luciferase, etc., but is not limited thereto. The drug resistance marker may be at least one selected from the group consisting of resistance genes to antibiotics (for example, ampicillin, streptomycin, gentamycin, kanamycin, hygromycin, tetracycline, chloramphenicol, neomycin, blasticidin, zeocin, puromycin, etc.), but is not limited thereto. The metabolism-related marker may be at least one selected from the group consisting of a thymidine kinase (TK) gene, a dihydrofolate reductase (DHFR) gene, and a glutamine synthetase (GS) gene, but is not limited thereto.

The reporter gene may be any gene that is typically used for selecting a cell having a specific DNA anchored thereat. For example, the reporter gene may be a lacZ reporter gene for facilitating the blue/white selection of transfected colonies, but is not limited thereto.

In an embodiment, the donor DNA structure may comprise a selection marker, an exogenous promoter, an SV40 intron, a signal peptide, and a tag sequentially in the 5′ to 3′ direction (see FIG. 1). FIG. 1 is a schematic view illustrating a procedure of inserting the donor DNA structure into a specific site at the 5′ end of the initiation codon of a target gene with the aid of a target-specific endonuclease system.

In another embodiment, the donor DNA molecule may comprise an exogenous promoter and a tag gene. The exogenous promoter and the tag gene may be carried by respective recombinant vectors (that is, the donor DNA structure may comprise a recombinant vector carrying an exogenous promoter and a recombinant vector carrying a tag gene). In this regard, the exogenous promoter and the tag gene may be inserted at different positions in the genome of the host cell. For example, the exogenous promoter may be inserted at the above-mentioned position, that is, the predetermined position to the 5′ end of the initiation codon of the target gene in the genome of the host cell while the tag gene may be introduced (inserted) at the initiation codon or the termination codon of the target gene or the endogenous peptide cleavage site (a site at which a signal peptide for an endogenous polypeptide of interest is cleaved and separated from the polypeptide of interest or a cleavage site by self-processing). As such, the insertion positions of the exogenous promoter and the tag gene may be variously selected depending on kinds and purposes of the target gene. For example, the tag gene may be introduced to the initiation codon (5′ UTR) and/or termination codon (3′ UTR) to insert the tag to the N-end and/or the C-end. Alternatively, the tag gene may be introduced into an endogenous peptide cleavage site so that the tag is inserted into a site at which the polypeptide of interest expressed by the target gene is cleaved and processed by an endonuclease within the cell. According to an embodiment, a tag gene may be inserted behind the signal peptide starting from the initiation codon of the RELN gene. As such, two or more target-specific endonuclease systems which target respective sites different to each other may be used in order to introduce (insert) the exogenous promoter and the tag gene to different sites in the genome of the host cell.

The donor DNA structure may be introduced (delivered) into a host cell by various methods known in the art, including, but not limited to, microinjection, electroporation, DEAE-dextran treatment, lipofection, nanoparticle-mediated transfection, protein translocation domain (PTD)-mediated transfer, virus-mediated gene transfer, and PEG-mediated transfection.

In an embodiment, the target-specific endonuclease system and/or the donor DNA structure may be introduced into a host cell via a typical vector. The vector may be a viral vector. The viral vector may be derived from at least one selected from the group consisting of negative-strand RNA viruses (e.g., influenza viruses), such as retrovirus, adenovirus, parvovirus (e.g., adeno-associated virus (AAV)), corona virus, and orthomyxovirus; positive-strand RNA viruses such as rhabdoviruses (e.g., rabies and vesicular stomatitis viruses), paramyxoviruses (e.g., measles and Sendai viruses), alphavirus, and picornavirus; double-stranded DNA viruses such as herpes viruses (e.g., herpes simplex type 1 and type 2, Epstein-Barr virus, cytomegalovirus), and adenoviruses; and poxviruses (e.g., vaccinia, fowlpox, and canarypox)), but is not limited thereto.

As described herein, the insertion of an exogenous promoter to a specific site of a target gene encoding an endogenous polypeptide of interest in the genome of a host cell can increase an expression level of the polypeptide of interest by 20% or more (1.2 fold or greater), 30% or more (1.3 fold or greater), 40% or more (1.4 fold or greater), 50% or more (1.5 fold or greater), 60% or more (1.6 fold or greater), 70% or more (1.7 fold or greater), 80% or more (1.8 fold or greater), 90% or more (1.9 fold or greater), 100% or more (2 fold or greater), 110% or more (2.1 fold or greater), 120% or more (2.2 fold or greater), 130% or more (2.3 fold or greater), 140% or more (2.4 fold or greater), 150% or more (2.5 fold or greater), 200% or more (3 fold or greater), 300% or more (4 fold or greater), 400% or more (5 fold or greater), 500% or more (6 fold or greater), 600% or more (7 fold or greater), 700% or more (8 fold or greater), 800% or more (9 fold or greater), 900% or more (10 fold or greater), or 1000% or more (11 fold or greater), compared to the case where the exogenous promoter is not inserted.

### Advantageous Effects

The promoter replacement and tag insertion provided in the present disclosure is an approach that can surmount the limitation according to the size of a polypeptide of interest and can precisely insert a sequence of interest (exogenous promoter and tag) into a desired site through homology directed repair (HDR) mediated by a target-specific endonuclease (e.g., Cas9) to express an endogenous gene, whereby the polypeptide of interest can be stably overexpressed and easily purified irrespective of the presence or absence of factors that make recombination difficult, such as sizes or repeat sequences of genes. In addition, untranslated region (UTR) or introns as well as coding sequences are known to influence gene expression.

Taking advantage of the endogenous genetic context of the cells themselves in which proteins are expressed, the target-specific endonuclease system can be used to insert a gene tag and thus can maintain an optimal condition for gene expression.

### DESCRIPTION OF DRAWINGS

FIG. 1 is a schematic view illustrating a procedure of inserting a promoter and an antibody tag into the genome of a host cell for overexpressing an endogenous polypeptide of interest according to an embodiment.

FIG. 2 shows DNA mutation ratios obtained by gene editing with various sgRNAs that specifically bind to a specific target nucleic acid sequence.

FIGS. 3a to 3c show data of flow cytometry for human cells in which an exogenous overexpression promoter and a tag gen are inserted into RELN gene.

FIGS. 4a and 4b shows detection data of Reelin protein in culture media of human single cell lines in which an exogenous promoter and a tag gene are inserted into RELN gene (4a: western blotting; 4b: quantitative results).

FIG. 5 shows detection data (immune-precipitation) of Reelin protein in culture media of human cells in which an exogenous promoter and a tag gene are inserted into RELN gene

FIG. 6 is a cleavage map of pAAY-hygro-sfGFP plasmid.

### MODE FOR INVENTION

A better understanding of the present disclosure may be obtained through the following examples which are set forth to illustrate, but are not to be construed as limiting the present disclosure.

### Example 1: Construction of Donor DNA Structure for Inserting Exogenous Promoter to Endogenous RELN Gene in Human Cell

It is known that RELN gene (NG_011877.1), which encodes human Reelin protein (NP_005036.2), is impossible to express through genetic recombination in human cells because it is large in size (genomic 150 kb, cDNA 11 kb) and contains modulated repeat sequences. In order to surmount such a limitation, a tag and an overexpression promoter were inserted into an endogenous RELN gene with the aid of a CRISPR gene editing system, followed by isolation and purification or Reelin protein from a human cell line (HEK293 cells).

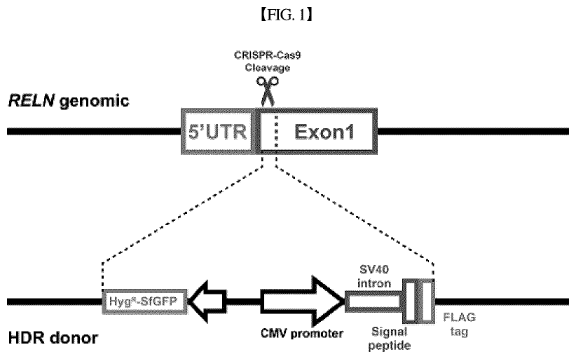

FIG. 1 is a schematic view illustrating the insertion of a donor DNA structure by means of a CRISPR gene editing system.

For overexpress the endogenous RELN gene, a CMV (Cytomegalovirus) promoter, which is an overexpression promoter, was inserted. A FLAG antibody tag was introduced downstream of the signal peptide encoding sequence Reelin (toward 3′ end) in order that when the signal peptide was cut out of the cell, the FLAG tag was exposed at the N-end and could be easy to detect and purify. In addition, a selection marker (Hyg^{R}-sfGFP) in which a hygromycin resistance gene (hygromycin phosphotransferase gene) and a super-fold green fluorescence protein (sfGFP) gene were combined to each other was inserted so as to easily discriminate cells having the gene introduced thereinto from the other cells. The template plasmid thus constructed was inserted behind the signal peptide starting from the initiation codon of the RELN gene. For constructing the template plasmid, pRG2 plasmid (purchased from ADDGENE) and pAAY-hygro-sfGFP plasmid (SEQ ID NO: 21 and FIG. 6) were employed.

The configuration of the donor DNA structure thus constructed is summarized in the 5′ to 3′ direction in Table 3, below:

TABLE 3

| Component | | Nucleic acid sequence (5′→3′) |
|---|---|---|
| Selection | Hyg^{R}_ | ATGAAAAAGCCTGAACTCACCGCGACGTCTGTCGAG |
| marker | sfGFP | AAGTTTCTGATCGAAAAGTTCGACAGCGTCTCCGAC |
| | | CTGATGCAGCTCTCGGAGGGCGAAGAATCTCGTGCT |
| | | TTCAGCTTCGATGTAGGAGGGCGTGGATATGTCCTG |
| | | CGGGTAAATAGCTGCGCCGATGGTTTCTACAAAGAT |
| | | CGTTATGTTTATCGGCACTTTGCATCGGCCGCGCTCC |
| | | CGATTCCGGAAGTGCTTGACATTGGGGAATTCAGCG |
| | | AGAGCCTGACCTATTGCATCTCCCGCCGTGCACAGG |
| | | GTGTCACGTTGCAAGACCTGCCTGAAACCGAACTGC |
| | | CCGCTGTTCTGCAGCCGGTCGCGGAGGCCATGGATG |
| | | CGATCGCTGCGGCCGATCTTAGCCAGACGAGCGGGT |
| | | TCGGCCCATTCGGACCGCAAGGAATCGGTCAATACA |
| | | CTACATGGCGTGATTTCATATGCGCGATTGCTGATCC |
| | | CCATGTGTATCACTGGCAAACTGTGATGGACGACAC |
| | | CGTCAGTGCGTCCGTCGCGCAGGCTCTCGATGAGCT |
| | | GATGCTTTGGGCCGAGGACTGCCCCGAAGTCCGGCA |
| | | CCTCGTGCACGCGGATTTCGGCTCCAACAATGTCCT |
| | | GACGGACAATGGCCGCATAACAGCGGTCATTGACTG |
| | | GAGCGAGGCGATGTTCGGGGATTCCCAATACGAGGT |
| | | CGCCAACATCTTCTTCTGGAGGCCGTGGTTGGCTTGT |
| | | ATGGAGCAGCAGACGCGCTACTTCGAGCGGAGGCA |
| | | TCCGGAGCTTGCAGGATCGCCGCGGCTCCGGGCGTA |
| | | TATGCTCCGCATTGGTCTTGACCAACTCTATCAGAGC |
| | | TTGGTTGACGGCAATTTCGATGATGCAGCTTGGGCG |
| | | CAGGGTCGATGCGACGCAATCGTCCGATCCGGAGCC |
| | | GGGACTGTCGGGCGTACACAAATCGCCCGCAGAAG |
| | | CGCGGCCGTCTGGACCGATGGCTGTGTAGAAGTACT |
| | | CGCCGATAGTGGAAACCGACGCCCCAGCACTCGTCC |
| | | GGATCGGGAGATGGGGGAGACTAGAATGTCTAAGG |
| | | TGGAACTGGATGGCGACGTGAACGGCCACAAGTTCT |
| | | CTGTGCGGGGAGAGGGCGAAGGCGACGCCACAAAT |
| | | GGCAAGCTGACCCTGAAGTTCATCTGCACCACCGGC |
| | | AAGCTGCCCGTGCCTTGGCCTACCCTCGTGACCACA |
| | | CTGACCTACGGCGTGCAGTGCTTCAGCAGATACCCC |
| | | GACCACATGAAGCGGCACGATTTCTTCAAGAGCGCC |
| | | ATGCCCGAGGGCTATGTGCAGGAACGGACCATCAGC |
| | | TTCAAGGACGACGGCACCTACAAGACCAGAGCCGA |
| | | AGTGAAGTTCGAGGGCGACACCCTCGTGAACCGGAT |
| | | CGAGCTGAAGGGCATCGACTTCAAAGAGGACGGCA |
| | | ACATCCTGGGCCACAAGCTGGAGTACAACTTCAACA |
| | | GCCACAACGTGTACATCACCGCCGACAAGCAGAAG |
| | | AACGGCATCAAGGCCAACTTCAAGATCCGGCACAAC |
| | | GTGGAAGATGGCAGCGTGCAGCTGGCCGACCACTAC |
| | | CAGCAGAACACCCCCATCGGAGATGGCCCCGTGCTG |
| | | CTGCCCGACAACCACTACCTGAGCACCCAGAGCGTG |
| | | CTGAGCAAGGACCCCAACGAGAAGCGGGACCACAT |
| | | GGTGCTGCTGGAATTTGTGACCGCCGCTGGCATCAC |
| | | CCACGGCATGGACGAGCTGTACAAGTCTAGTTGA |
| | | (SEQ ID NO: 9) |
| Promoter | CMV | TAGTTATTAATAGTAATCAATTACGGGGTCATTAGTT |
| | (Cytomegalovirus) | CATAGCCCATATATGGAGTTCCGCGTTACATAACTT |
| | promoter | ACGGTAAATGGCCCGCCTGGCTGACCGCCCAACGAC |
| | | CCCCGCCCATTGACGTCAATAATGACGTATGTTCCC |
| | | ATAGTAACGCCAATAGGGACTTTCCATTGACGTCAA |
| | | TGGGTGGAGTATTTACGGTAAACTGCCCACTTGGCA |
| | | GTACATCAAGTGTATCATATGCCAAGTACGCCCCCT |
| | | ATTGACGTCAATGACGGTAAATGGCCCGCCTGGCAT |
| | | TATGCCCAGTACATGACCTTATGGGACTTTCCTACTT |
| | | GGCAGTACATCTACGTATTAGTCATCGCTATTACCAT |
| | | GGTGATGCGGTTTTGGCAGTACATCAATGGGCGTGG |
| | | ATAGCGGTTTGACTCACGGGGATTTCCAAGTCTCCA |
| | | CCCCATTGACGTCAATGGGAGTTTGTTTTGGCACCA |
| | | AAATCAACGGGACTTTCCAAAATGTCGTAACAACTC |
| | | CGCCCCATTGACGCAAATGGGCGGTAGGCGTGTACG |
| | | GTGGGAGGTCTATATAAGCAGAGCTGGTTTAGTGAA |
| | | CCGTCAGAT (SEQ ID NO: 10) |
| | SV40 | GTAAGTATCAAGGTTACAAGACAGGTTTAAGGAGAC |
| | intron | CAATAGAAACTGGGCTTGTCGAGACAGAGAAGACT |
| | | CTTGCGTTTCTGATAGGCACCTATTGGTCTTACTGAC |
| | | ATCCACTTTGCCTTTCTCTCCACAG (SEQ ID NO: 11) |
| | Signal | ATGGAGCGCAGTGGCTGGGCCCGGCAGACTTTTCTC |
| | peptide | CTAGCGCTGTTGCTGGGGGCGACGCTGAGAGCGCGC |
| | | GCG (SEQ ID NO: 12) |
| Tag | FLAG tag | GATTACAAGGATGACGATGACAAG (SEQ ID NO: 13) |

### Example 2: Selection of Target Nucleic Acid Sequence for Inserting Exogenous Promoter into Endogenous RELN Gene of Human Cell

The donor DNA structure prepared in Example 1 was inserted into the genome of human host cells (HEK293E or HEK293 c18 cell; ATCC #CRL-10852). To this end, a Cas9 system including six single-guide RNAs (sgRNAs) was first established. From them, sgRNAs with high efficiency were selected. Selection was made of sgRNAs such that parts of nucleic acid sequences in the vicinity of the initiation codon of the RELN gene were adopted as target nucleic acid sequences when they were free of off-targets under the condition of tolerating up to 10 two mismatches. The results are summarized in Table 4, below.

TABLE 4

| Guide | RGEN Target sequence | SEQ |
|---|---|---|
| RNA | (5′ to 3′) | ID NO |
| RELN_sg1 | GCGCTAGGAGGAAAGTCTGCCGG | 14 |
| RELN_sg2 | TTTCCTCCTAGCGCTGTTGCTGG | 15 |
| RELN_sg3 | TTCCTCCTAGCGCTGTTGCTGGG | 16 |
| RELN_sg4 | TCCTCCTAGCGCTGTTGCTGGGG | 17 |
| RELN_sg5 | CCCCCAGCAACAGCGCTAGGAGG | 18 |
| RELN_sg6 | TGTTGCTGGGGGCGACGCTGAGG | 19 |
| RELN_sg7 | GTTGCTGGGGGCGACGCTGAGGG | 20 |

The sgRNA had the following nucleic acid sequence:

5′-(targeting sequence)-(GUUUUAGAGCUA; SEQ ID NO: 1)-(nucleotide linker)-
(UAGCAAGUUAAAAUAAGGCUAGUCCGUUAUCAACUUGAAAAAGUGGCACCG
AGUCGGUGC; SEQ ID NO: 3)-3′

(the targeting sequence is complementary to the target nucleic acid sequence of the complementary strand to a strand on which the PAM sequence is located, and has the same sequence as the RGEN Target sequence of Table 3 minus the 3′ end PAM sequence when T″ is converted to “U”, and the nucleotide linker has the nucleotide sequence of GAAA).

The selected target nucleic acid sequences were evaluated for efficiency. In this regard, first, a DNA coding for sgRNA (RELN_sg1 to RELN_sg7) including a targeting sequence for the target nucleic acid sequence was inserted into pRG2 plasmid (purchased from ADDGENE) to prepare an sgRNA expression plasmid. Together with 750 ng of Cas9 expression p3S-Cas9HC plasmid (purchased from ADDGENE), 250 ng of the sgRNA expression plasmid was transfected into 1×10⁵ HEK293E cells (ATCC #CRL-10852) by lipofection using Lipofectamine 2000 (Invitrogen).

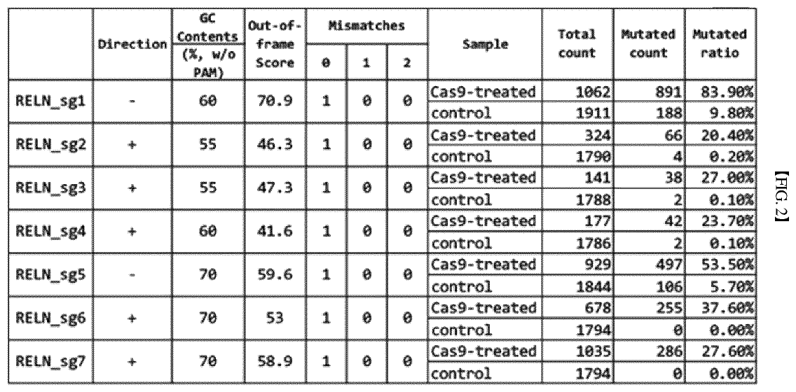

% DNA mutation of the targeting region (%; [mutated count/total count]*100) was measured by targeted deep-sequencing using NGS (see Jeongbin Park, Kayeong Lim, Jin-Soo Kim, Sangsu Bae; Cas-analyzer: an online tool for assessing genome editing results using NGS data, Bioinformatics, Volume 33, Issue 2, 15 Jan. 2017, Pages 286-288) and the measurements are given in FIG. 2. Among the sgRNAs tested, as shown in FIG. 2, RELN_sg1, RELN_sg5, and RELN_sg6 exhibit high efficiencies. Of them, selection was made of RELN_sg5 and RELN_sg6 which were used in the following experiments.

### Example 3: Insertion of Exogenous Promoter into Endogenous RELN Gene in Human Cell

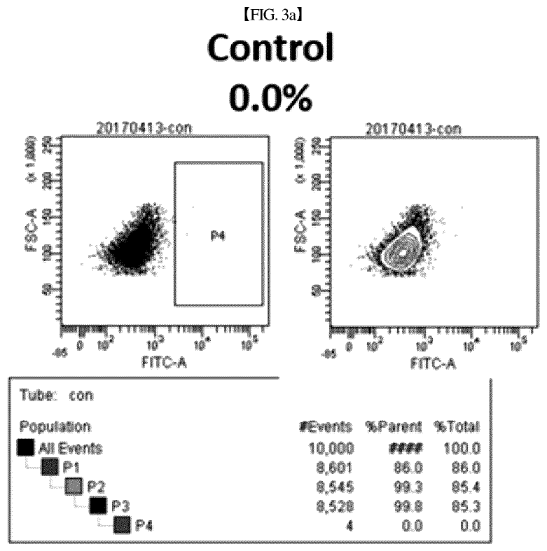

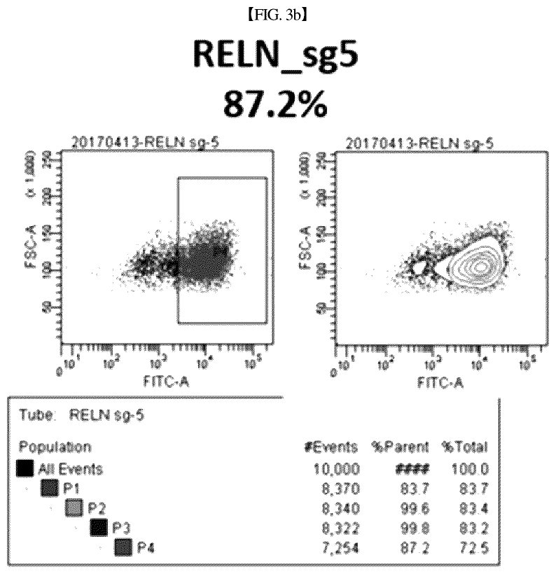

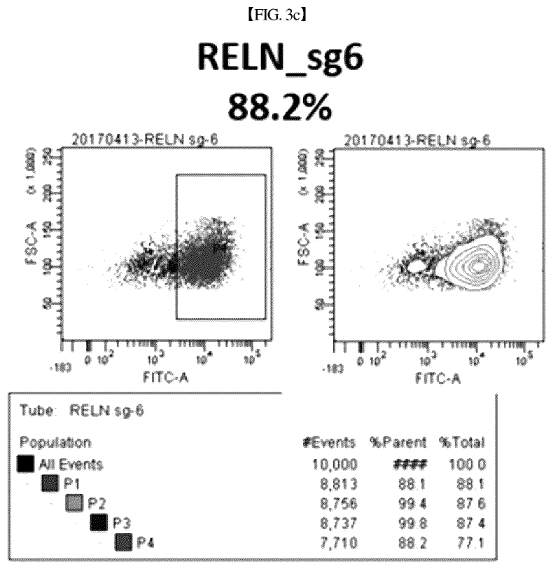

Cas9-sgRNA (RELN_sg5, sg6) selected in Example 2 and the donor DNA structure prepared in Example 1 were transfected into HEK293E cells to induce gene insertion through homology directed repair (HDR). Briefly, 250 ng of sgRNA expression plasmid, 750 ng of Cas9 expression plasmid, and 1000 ng of the donor DNA structure of Example 1 were transfected into HEK293E cells (ATCC #CRL-10852) by Lipofection and the cells were incubated at 37° ° C. in DMEM (10% FBS, 1% antibiotics/WELGENE) in a 5% CO₂ atmosphere. In order to select transfected cells, hygromycin was added to the cell culture to allow only the gene-inserted cells to selectively survive. After being cultured for about two weeks, the cells were assayed by flow cytometry (FACS) to separate only the cells exhibiting the fluorescent marker GFP signal. The results are given in FIGS. 3a to 3c. In FIGS. 3a to 3c, P1 stands for a total count of cells subjected to flow cytometry, P2 for a count of cells that are analyzed as single normal cells in P1 through the cell size assay, P3 for a count of cells analyzed as viable cells, P4 for a count of cells exhibiting GFP fluorescence in P3 (that is, a count of cells having a desired donor inserted thereinto), #Event for a number of analysis (cell count), % parent for a ratio of Pn+1 to Pn, and % total for a ratio of Pn to all events. The HEK293E cells into which the donor DNA structure including the exogenous promoter was inserted was obtained by separating cells from which the fluorescent marker was detected (exogenous promoter inserted) at high yield (P4 (% Parent) about 87-88%) because primary selection against hygromycin was performed.

### Example 4: Overexpression of Reelin Protein in Human Cell Having Exogenous Promoter Inserted Thereinto and Establishment of Homogenous Cell Line

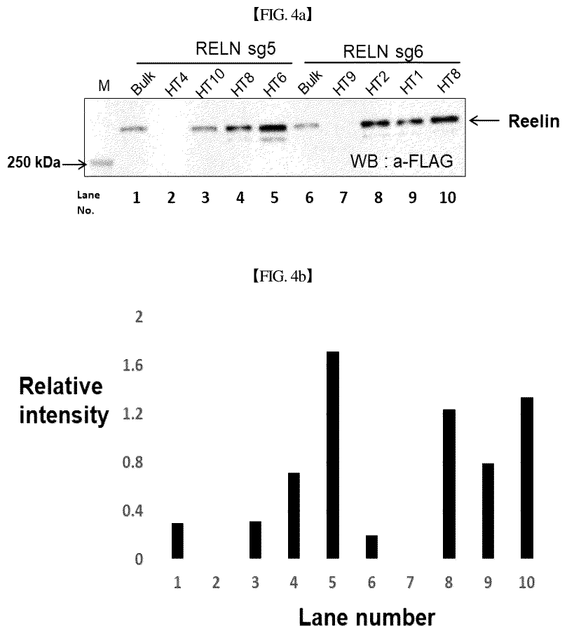

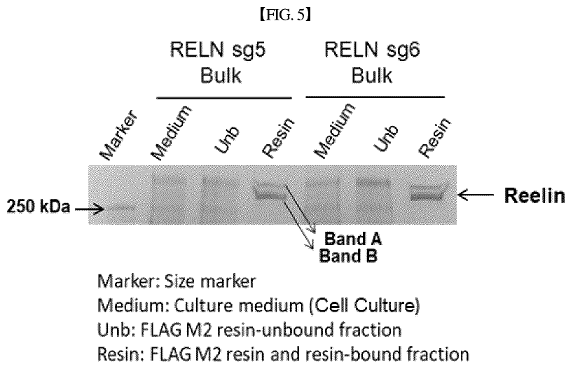

In order to examine whether the human cells into which the donor DNA structure, constructed in Example 3, comprising the exogenous promoter was inserted expressed the Reelin protein containing the tag, western blotting was performed using a FLAG M2 antibody. In brief, the HEK293E cells into which the donor DNA structure containing the exogenous promoter was inserted as obtained in Example 3 were cultured at 37° C. in DMEM (10% FBS, 1% antibiotics/WELGENE) under a 5% CO₂ condition. The cell culture thus obtained were incubated with FLAG M2 antibody (MERCK F3165), followed by western blotting. The results are depicted in FIG. 4.

In detail, the establishment of a homogenous cell line started with single cell culturing. The HEK293E cells, obtained in Example 3, having the exogenous promoter-containing donor DNA structure inserted thereinto were plated at a density of one cell per well to 96-well plates containing DMEM (10% FBS, 1% antibiotics/WELGENE) medium and cultured at 37° C. for three weeks in a 5% CO₂ atmosphere. The cell cultures were treated with FLAG M2 antibody (MERCK F3165) as described above, followed by western blotting. The result is given in FIG. 4a and the quantitative data are depicted in FIG. 4b. In FIGS. 4a and 4b, HT-serial numbers stand for single cell lines cultured in respective wells, M for culture medium, and Bulk for a cluster of 1×106 HEK293E cells, obtained in Example 3, having the exogenous promoter-containing donor DNA structure inserted thereinto. As can be seen in FIGS. 4a and 4b, very high Reelin expression and secretion levels were detected in the two single cell lines RELN_sg5-HT6 and RELN_sg6-HT8, compared to the bulk cells. Data of FIGS. 4a and 4b show that relatively much amounts of the Reelin protein tagged with FLAG were detected in the culture media of the cells, indicating that Reelin protein was expressed and well secreted out of the cells.

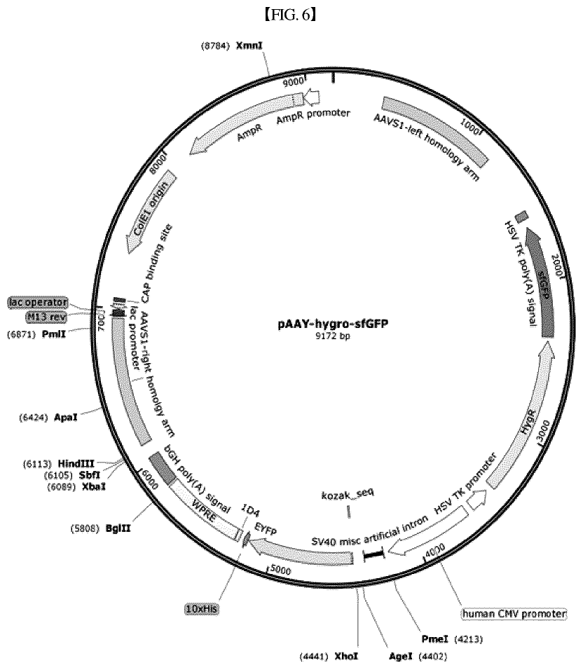

To test whether or not the purification of Reelin protein was possible, immune-precipitation was applied to a small amount of the sample. RELN_sg5 Bulk and RELN_sg6 Bulk cell cultures were each added in an amount of 1.4 mL to respective 1.5 mL-tubes and mixed with 10 μL of FLAG M2 antibody resins. Mixing was carried out by slowly rotating the tubes for one hour. Centrifugation separated the resins and resin-bound fractions from resin-unbound fractions, followed by SDS-PAGE analysis. The result is depicted. As shown in FIG. 5, 1) culture medium, 2) resin-unbound protein fraction, 3) resin and resin-bound protein fraction were loaded in that order and run. The SDS-PAGE data of FIG. 5 show that the Reelin protein could be purified from both the RELN_sg5 Bulk and RELN_sg6 Bulk cell culture media as the protein were well bound to the FLAG M1 antibody resins.

Additionally, the FLAG M1 antibody resin-bound protein from the RELN_sg5 Bulk cell culture medium was subjected to mass spectrometry (LC-MS). The two protein bands observed in FIG. 5 (Band A and Band B) were analyzed and mass spectrum data of the purified proteins are given in Tables 5 and 6, below.

TABLE 5
Mass analysis results of proteins purified from Band A

| Protein | Peptide | Sum of Peptide spectrum |
|---|---|---|
| RELN_HUMAN | 104 | 359 |
| sp\|P00761\|TRYP_PIG | 2 | 15 |
| HS90B_HUMAN | 3 | 8 |
| EF1A1_HUMAN | 3 | 8 |
| G3P_HUMAN | 3 | 7 |
| VIME_HUMAN | 3 | 7 |
| TBA1A_HUMAN | 3 | 6 |
| TCPH_HUMAN | 5 | 6 |
| EF2_HUMAN | 3 | 6 |
| NPM_HUMAN | 1 | 5 |

TABLE 6
Mass analysis results of proteins purified from Band B

| Protein | Peptide | Sum of Peptide spectrum |
|---|---|---|
| RELN HUMAN | 116 | 580 |
| sp\|P00761\|TRYP_PIG | 2 | 16 |
| ALBU_HUMAN | 4 | 8 |
| SVEP1_HUMAN | 7 | 7 |
| VIME_HUMAN | 2 | 6 |
| EF2_HUMAN | 4 | 6 |
| G3P_HUMAN | 3 | 6 |
| EF1A1_HUMAN | 3 | 5 |
| HNRH1_HUMAN | 2 | 5 |
| ACTA_HUMAN | 4 | 5 |

As understood from the data of Tables 5 and 6, the two bands were both observed to have the sequence of human Reelin protein (RELN_HUMAN).

As described above, the mass production of Reelin can be achieved by culturing the single cell lines with a high potential of Reelin expression on a mass scale.

Reelin is a secreted protein that is involved in brain development and neuronal regulation. In spite of the importance thereof, the protein has not been successfully produced and extracted through recombination due to the huge size thereof and the presence of repeat sequences therein, thus far. For this reason, the precise structure of Reelin remains unknown.

The promoter replacement and tag insertion for an endogenous gene by use of the target-specific endonuclease system provided in the present disclosure is an approach that can push through such impasses. The target-specific endonuclease system can precisely insert a desired sequence into a desired site through homology directed repair (HDR) mediated by a target-specific endonuclease (e.g., Cas9). In addition, the target-specific endonuclease system is not affected at all by factors that make recombination difficult, such as sizes or repeat sequences of genes because the system is designed to express endogenous genes.

In addition, untranslated region (UTR) or introns as well as coding sequences (CDS) are known to influence gene expression. Taking advantage of the endogenous genetic context of the cells themselves in which proteins are expressed, the target-specific endonuclease system can maintain an optimal condition for gene expression.

1-34. (canceled)

35. A method of expressing or producing a polypeptide of interest in an animal cell, the composition, the method comprising a step of introducing the composition for expressing a polypeptide of interest into the animal cell, wherein
the composition comprises a target-specific endonuclease system and a donor DNA structure,
the polypeptide of interest is an endogenous polypeptide encoded by a genome of the animal cell,
the target-specific endonuclease system comprises: a target-specific endonuclease, a first nucleic acid molecule coding therefor, or a first recombinant vector comprising the first nucleic acid molecule; and a nucleic acid molecule (a second nucleic acid molecule) capable of hybridizing with a target nucleic acid sequence adjacent to a 5′ end side of a gene encoding the endogenous polypeptide of interest or a second recombinant vector comprising the second nucleic acid molecule, and
the donor DNA structure comprises an exogenous promoter derived from a cell heterogenous to the animal cell.

36. The method of claim 35, wherein the target-specific endonuclease is at least one selected from the group consisting of TALEN (transcription activator-like effector nuclease), zinc-finger nuclease (ZFN), RNA-guided endonuclease (RGEN), and DNA-guided endonuclease.

37. The method of claim 35, wherein the target-specific endonuclease is RNA-guided endonuclease (RGEN), and the second nucleic acid molecule is a guide RNA capable of hybridizing with a target nucleic acid sequence adjacent to a 5′ end side of a gene encoding the endogenous polypeptide of interest.

38. The method of claim 37, wherein the target-specific endonuclease is at least one selected from group consisting of Cas9 proteins derived from Streptococcus pyogenes, Campylobacter jejuni, Streptococcus thermophiles, Streptocuccus aureus, Neisseria meningitidis, Pasteurella multocida, and Francisella novicida.

39. The method of claim 35, wherein the donor DNA structure further comprises a tag gene.

40. The method of claim 39, wherein the donor DNA structure further comprises a nucleic acid molecule encoding a signal peptide for the endogenous polypeptide of interest at a 5′ end of the tag gene.

41. The method of claim 40, wherein the target-specific endonuclease system cleaves the 3′ end or an inner site of the nucleic acid sequence encoding the signal peptide.

42. The method of claim 35, wherein the polypeptide of interest contains 2 to 10,000 amino acids.

43. The method of claim 35, wherein the target-specific endonuclease system cleaves a site between an initial codon and an endogenous promoter or 5′-UTR in a gene coding for the polypeptide of interest.

44. The method of claim 35, wherein the animal cell is a mammalian cell.

45. A recombinant cell, prepared by introducing a donor DNA structure into a host cell, wherein the donor DNA structure is inserted to a 5′ end side of a gene coding for an endogenous polypeptide of interest within a genome of the host cell, and comprises an exogenous promoter derived from a cell heterogenous to the host cell, the host cell being an animal cell.

46. The recombinant cell of claim 45, wherein the donor DNA structure further comprises a tag gene.

47. The recombinant cell of claim 46, wherein the donor DNA structure further comprises a nucleic acid molecule encoding a signal peptide for the endogenous polypeptide of interest at a 5′ end of the tag gene.

48. The recombinant cell of claim 45, wherein the endogenous polypeptide of interest contains 2 to 10,000 amino acids.

49. The recombinant cell of claim 45, wherein the donor DNA structure is inserted between an initial codon and an endogenous promoter or 5′-UTR in a gene coding for the polypeptide of interest within a genome of the host cell.

50. The recombinant cell of claim 45, wherein the host cell is a mammalian cell.

51. A method for producing a polypeptide of interest in an animal cell, the method comprising a step of culturing the recombinant cell of claim 45.

52. The method of claim 51, wherein the donor DNA structure further comprises a tag gene, and a nucleic acid molecule encoding a signal peptide for the endogenous polypeptide of interest at a 5′ end of the tag gene.

53. The method of claim 51, further comprising isolating or purifying the polypeptide of interest after the culturing step.
